(19) United States
(12) Patent Application Publication
(10) Pub. No.: US 2008/0241225 A1
(43) Pub. Date: Oct. 2, 2008

(54) BASIC PROCESSES TO PREPARE ANTIMICROBIAL CONTACT LENSES

(76) Inventors: Gregory A. Hill, Atlantic Beach, FL (US); Kent Young, Jacksonville, FL (US); Hassan Chaouk, Jacksonville, FL (US); Osman Rathore, Jacksonville, FL (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/052,795

(22) Filed: Mar. 21, 2008

Related U.S. Application Data
(60) Provisional application No. 60/921,029, filed on Mar. 31, 2007.

(51) Int. Cl. A61K 9/08; B29D 11/00

(52) U.S. Cl. 424/429; 264/2.6

(57) ABSTRACT

This invention relates to antimicrobial lenses containing metals and methods for their production.

### RELATED APPLICATION

This application is a non-provisional filing of a provisional application, U.S. Ser. No. 60/921,029, filed on Mar. 31, 2007.

### FIELD OF THE INVENTION

This invention relates to methods of preparing antimicrobial lenses.

### BACKGROUND OF THE INVENTION

Contact lenses have been used commercially to improve vision since the 1950s. The first contact lenses were made of hard materials. They were used by a patient during waking hours and removed for cleaning. Current developments in the field gave rise to soft contact lenses, which may be worn continuously, for several days or more without removal for cleaning. Although many patients favor these lenses due to their increased comfort, these lenses can cause some adverse reactions to the user. The extended use of the lenses can encourage the buildup of bacteria or other microbes, particularly, Pseudomonas aeruginosa, on the surfaces of soft contact lenses. The build-up of bacteria and other microbes can cause adverse side effects such as contact lens acute red eye and the like. Although the problem of bacteria and other microbes is most often associated with the extended use of soft contact lenses, the build-up of bacteria and other microbes occurs for users of hard contact lens wearers as well.

Others have taught that the addition of antibacterial agents such as metal salts to contact lenses can inhibit the growth of bacteria or other microbes. See, US 2004/0150788, which is hereby incorporated by reference in its entirety. When antibacterial agents are incorporated into lenses, a lens that is cloudy or hazy lens may be produced. The level of haze often increases when the content of antibacterial agents in the lens increase. This haze is though to be caused by the clustering of antibacterial agents in the lens. Unfortunately this haze can obscure a user's vision and may even be visible to the user upon inspection. Since neither of these conditions is desirable, it would be beneficial if one could incorporate larger amounts of antibacterial agents into a lens with a minimal amount of haze. This need are met by the following invention.

### DETAILED DESCRIPTION OF THE INVENTION

This invention includes a method of preparing an ionic antimicrobial lens comprising a metal salt, wherein said method comprising the steps of
(a) treating an cured ionic lens for a sufficient period of time, with a first solution wherein the pH of said first solution is equal to the pKa of the ionic monomers, that were cured to form said ionic lens;
(b) adding a metal agent to said first solution and said cured lens after step (a)
(c) treating the lens of step (b) with a second solution comprising a salt precursor.
   As used herein, the term, “antimicrobial lens” means a lens that exhibits one or more of the following properties, the inhibition of the adhesion of bacteria or other microbes to the lenses, the inhibition of the growth of bacteria or other microbes on lenses, and the killing of bacteria or other microbes on the surface of lenses or in an area surrounding the lenses. For purposes of this invention, adhesion of bacteria or other microbes to lenses, the growth of bacteria or other microbes on lenses and the presence of bacteria or other microbes on the surface of lenses are collectively referred to as “microbial colonization.” Preferably, the lenses of the invention exhibit a reduction of viable bacteria or other microbe of at least about 0.25 log, more preferably at least about 0.5 log, most preferably at least about 1.0 log (≧90% inhibition). Such bacteria or other microbes include but are not limited to those organisms found in the eye, particularly Pseudomonas aeruginosa, Acanthamoeba species, Staphylococcus aureus, Escherichia coli, Staphylococcus epidermidis, and Serratia marcesens.

As used herein, the term “lens” refers to an ophthalmic device that resides in or on the eye. These devices can provide optical correction, wound care, drug delivery, diagnostic functionality, cosmetic enhancement or effect, or any combination of these properties. The term lens includes but is not limited to soft contact lenses, hard contact lenses, intraocular lenses, overlay lenses, ocular inserts, and optical inserts. Soft contact lenses are made from silicone elastomers or hydrogels, which include but are not limited to silicone hydrogels, and fluorohydrogels.

Lenses of the invention may be made from silicone hydrogel components. A silicone-containing component is one that contains at least one [—Si—O—Si] group, in a monomer, macromer or prepolymer. Preferably, the Si and attached O are present in the silicone-containing component in an amount greater than 20 weight percent, and more preferably greater than 30 weight percent of the total molecular weight of the silicone-containing component. Useful silicone-containing components preferably comprise polymerizable functional groups such as acrylate, methacrylate, acrylamide, methacrylamide, N-vinyl lactam, N-vinylamide, and styryl functional groups. Examples of silicone components which may be included in the silicone hydrogel formulations include, but are not limited to silicone macromers, prepolymers and monomers. Examples of silicone macromers include, without limitation, polydimethylsiloxane methacrylated with pendant hydrophilic groups as described in U.S. Pat. Nos. 4,259,467; 4,260,725 and 4,261,875; polydimethylsiloxane macromers with polymerizable functional group(s) described in U.S. Pat. Nos. 4,136,250; 4,153,641; 4,189,546; 4,182,822; 4,343,927; 4,254,248; 4,355,147; 4,276,402; 4,327,203; 4,341,889; 4,486,577; 4,605,712; 4,543,398; 4,661,575; 4,703,097; 4,837,289; 4,954,586; 4,954,587; 5,346,946; 5,358,995; 5,387,632; 5,451,617; 5,486,579; 5,962,548; 5,981,615; 5,981,675; and 6,039,913; polysiloxane macromers incorporating hydrophilic monomers such as those described in U.S. Pat. Nos. 5,010,141; 5,057,578; 5,314,960; 5,371,147 and 5,336,797; macromers comprising polydimethylsiloxane blocks and polyether blocks such as those described in U.S. Pat. Nos. 4,871,785 and 5,034,461, combinations thereof and the like. All of the patents cited herein are hereby incorporated in their entireties by reference.

The silicone and/or fluorine containing macromers described in U.S. Pat. Nos. 5,760,100; 5,776,999; 5,789,461; 5,807,944; 5,965,631 and 5,958,440 may also be used. Suitable silicone monomers include tris(trimethylsiloxy)silylpropyl methacrylate, hydroxyl functional silicone containing monomers, such as 3-methacryloxy-2-hydroxypropyloxy)propylbis(trimethylsiloxy)methylsilane and those disclosed in WO03/22321, and mPDMS containing or the siloxane monomers described in U.S. Pat. Nos. 4,120,570, 4,139,692, 4,463,149, 4,450,264, 4,525,563; 5,998,498; 3,808,178; 4,139,513; 5,070,215; 5,710,302; 5,714,557 and 5,908,906.

Additional suitable siloxane containing monomers include, amide analogs of TRIS described in U.S. Pat. No. 4,711,943, vinylcarbamate or carbonate analogs described in U.S. Pat. No. 5,070,215, and monomers contained in U.S. Pat. No. 6,020,445, monomethacryloxypropyl terminated polydimethylsiloxanes, polydimethylsiloxanes, 3-methacryloxypropylbis(trimethylsiloxy)methylsilane, methacryloxypropylpentamethyl disiloxane and combinations thereof.

“Ionic lenses” are lens formulations that contain “ionic monomers.” Examples of ionic monomer include but are not limited to methacrylic acid, acrylic acid, styrene sulfonate, 2-acrylamido-2-methylpropane sulfonic acid, and 2-methacryloyloxyethyl phosphorylcholine. Examples of ionic lenses include but are not limited to the Group III and Group IV lenses as those terms are defined by the US Food and Drug Administration. Preferred ionic lenses are selected from the group consisting of etafilcon A, balafilcon A, bufilcon A, deltafilcon A, droxifilcon A, phemfilcon A, ocufilicon A, perfilcon A, ocufilcon B, ocufilcon C, ocufilcon D, ocufilcon E, metafilcon A, B, vifilcon A focofilcon A, and tetrafilcon B.

Preferably, the lenses of the invention are optically clear, with optical clarity comparable to lenses such as lenses made from etafilcon A, genfilcon A, galyfilcon A, lenefilcon A, polymacon, acquafilcon A, balafilcon A, and lotrafilcon A.

Many of the lens formulations cited above may allow a user to insert the lenses for a continuous period of time ranging from one day to thirty days. It is known that the longer a lens is on the eye, the greater the chance that bacteria and other microbes will build up on the surface of those lenses. Therefore, an advantage of the methods of the invention is that one can add more metal salt to the lenses with reduced haze.

As use herein, the term “metal salt” means any molecule having the general formula [M]ₐ [X]_{b} wherein X contains any negatively charged ion, a is ≧1, b is ≧1 and M is any positively charged metal selected from, but not limited to, the following Al⁺³, Co⁺², Co⁺³, Ca⁺², Mg⁺², Ni⁺², Ti⁺², Ti⁺³, Ti⁺⁴, V⁺², V⁺³, V⁺⁵, Sr⁺², Fe⁺², Fe⁺³, Ag⁺¹, Ag⁺², Au⁺², Au⁺³, Au⁺¹, Pd⁺², Pd⁺⁴, Pt⁺², Pt⁺⁴, Cu⁺¹, Cu⁺², Mn⁺², Mn⁺³, Mn⁺⁴, Zn⁺², and the like. Examples of X include but are not limited to CO₃⁻², NO₃⁻¹, PO₄⁻³, Cl⁻¹, I⁻¹, Br⁻¹, S⁻², O⁻² and the like. Further X includes negatively charged ions containing CO₃⁻² NO₃⁻¹, PO₄⁻³, Cl⁻¹, I⁻¹, Br⁻¹, S⁻², O⁻², and the like, such as C₁₋₅alkylCO₂⁻¹. As used herein the term metal salts does not include zeolites, disclosed in WO03/011351. This patent application is hereby incorporated by reference in its entirety. The preferred a is 1, 2, or 3. The preferred b is 1, 2, or 3. The preferred metals ions are Mg⁺², Zn⁺², Cu⁺¹, Cu⁺², Au⁺², Au⁺³, Au⁺¹, Pd⁺², Pd⁺⁴, Pt⁺², Pt⁺⁴, Ag⁺², and Ag⁺¹. The particularly preferred metal ion is Ag⁺¹. Examples of suitable metal salts include but are not limited to manganese sulfide, zinc oxide, zinc sulfide, copper sulfide, and copper phosphate. Examples of silver salts include but are not limited to silver nitrate, silver sulfate, silver iodate, silver carbonate, silver phosphate, silver sulfide, silver chloride, silver bromide, silver iodide, and silver oxide. The preferred silver salts are silver iodide, silver chloride, and silver bromide.

The amount of metal in the lenses is measured based upon the total weight of the lenses. When the metal is silver, the preferred amount of silver is about 0.00001 weight percent (0.1 ppm) to about 10.0 weight percent, preferably about 0.0001 weight percent (1 ppm) to about 1.0 weight percent, most preferably about 0.001 weight percent (10 ppm) to about 0.1 weight percent, based on the dry weight of the lens. With respect to adding metal salts, the molecular weight of the metal salts determines the conversion of weight percent of metal ion to metal salt. The preferred amount of silver salt is about 0.00003 weight percent (0.3 ppm) to about 50.0 weight percent, preferably about 0.0003 weight percent (3 ppm) to about 5.0 weight percent, most preferably about 0.003 weight percent (30 ppm) to about 0.5 weight percent, based on the dry weight of the lens.

The term “salt precursor” refers to any compound or composition (including aqueous solutions) that contains a cation that may be substituted with metal ions. The concentration of salt precursor in its solution is between about 0.00001 to about 10.0 weight percent, (0.1-100,000 ppm) more preferably about 0.0001 to about 1.0 weight percent, (1-10,000 ppm) most preferably about 0.001 to about 0.1 weight percent (10-1000 ppm) based upon the total weight of the solution. Examples of salt precursors include but are not limited to inorganic molecules such as sodium chloride, sodium iodide, sodium bromide, sodium sulfide, lithium chloride, lithium iodide, lithium bromide, lithium sulfide, potassium bromide, potassium chloride, potassium sulfide, potassium iodide, rubidium iodide, rubidium bromide, rubidium chloride, rubidium sulfide, caesium iodide, caesium bromide, caesium chloride, caesium sulfide, francium iodide, francium bromide, francium chloride, francium sulfide, sodium tetrachloro argentite, and the like. Examples of organic molecules include but are not limited to tetra-alkyl ammonium lactate, tetra-alkyl ammonium sulfate, quaternary ammonium halides, such as tetra-alkyl ammonium chloride, bromide or iodide. The preferred salt precursor is selected from the group consisting of sodium chloride, sodium iodide, sodium bromide, lithium chloride, lithium sulfide, sodium sulfide, potassium sulfide, potassium iodide, and sodium tetrachloro argentite and the particularly preferred salt precursor is sodium iodide.

The term “metal agent” refers to any composition (including aqueous solutions) containing metal ions. Examples of such compositions include but are not limited to aqueous or organic solutions of silver nitrate, silver triflate, or silver acetate, silver tetrafluoroborate, silver sulfate, zinc acetate, zinc sulfate, copper acetate, and copper sulfate, where the concentration of metal agent in solution is about 1 μg/mL or greater. The preferred metal agent is aqueous silver nitrate, where the concentration of silver nitrate is the solution is about greater than or equal to 0.0001 to about 2 weight percent (1-20,000 ppm), more preferably about greater than 0.001 to about 0.2 weight percent (10-2000 ppm), more preferably about 0.01 to about 0.2 weight percent (100-2000 ppm), based on the total weight of the solution.

The term “solution” refers to an aqueous substance such as deionized water, saline solutions, borate or buffered saline solution, or organic substance such as C1-C24 alcohols, cyclic amides, acyclic amides, ethers and acids. The pH of the solution may be adjusted by adjusting the amount of basic components (i.e. borate) of said solutions. While not wishing to be bound by a particular methodology, it is believed that the methods of the invention de-protinate the pendant groups which impart ionicity to the ionic lenses. For example if a lens formulation is made from the ionic monomer methacrylic acid, the pendant ionic group is the carboxylate group of methacrylic acid. The pH of the first solution is determined by the pKa of the ionic monomers in the ionic lens. It is preferred that the pH of the first solution is above the pKa of the ionic monomer. Preferably the pH of the first solution about 1 unit greater than the pKa of the ionic monomers, more preferably at least about 2 units greater, even more preferably at least about 2 units greater to about 4 units greater. For example if the ionic monomer is methacrylic acid, preferably the pH of the first solution is about pH 5 to about pH 9, more preferably about pH 6 to about pH 8.

The term “treating” refers to any method of contacting solutions of the metal agent and the salt precursor with the cured lens, where the preferred method is immersing the lens in a solution of containing either the metal agent or the salt precursor. Treating can include heating the lens in these solutions, but it preferred that treating is carried out at ambient temperatures. The time of treating is preferably about 1 minute to about 24 hours. The treating time for the first solution is preferably longer than the treating time for the second solution. For example the treating time of the first solution may be from about 4 hours to about 16 hours and the treating time of the second solution may be from about 1 minute to about 10 minutes.

The term “cured” refers to any of a number of methods used to react a mixture of lens components (i.e., monomer, prepolymers, macromers and the like) to form lenses. Lenses can be cured by light or heat. The preferred method of curing is with radiation, preferably UV or visible light, and most preferably with visible light. The lens formulations of the present invention can be formed by any of the methods know to those skilled in the art, such as shaking or stirring, and used to form polymeric articles or devices by known methods. For example, the antimicrobial lenses of the invention may be prepared by mixing reactive components and any diluent(s) with a polymerization initator and curing by appropriate conditions to form a product that can be subsequently formed into the appropriate shape by lathing, cutting and the like. Alternatively, the reaction mixture may be placed in a mold and subsequently cured into the appropriate article.

Various processes are known for processing the lens formulation in the production of contact lenses, including spincasting and static casting. Spincasting methods are disclosed in U.S. Pat. Nos. 3,408,429 and 3,660,545, and static casting methods are disclosed in U.S. Pat. Nos. 4,113,224 and 4,197,266. The preferred method for producing antimicrobial lenses of this invention is by molding. In the case of hydrogel lenses, for this method, the lens formulation is placed in a mold having the approximate shape of the final desired lens, and the lens formulation is subjected to conditions whereby the components polymerize, to produce a hardened disc that is subjected to a number of different processing steps including treating the polymerized lens with liquids (such as water, inorganic salts, or organic solutions) to swell, or otherwise equilibrate this lens prior to enclosing the lens in its final packaging. This method is further described in U.S. Pat. Nos. 4,495,313; 4,680,336; 4,889,664; and 5,039,459, incorporated herein by reference. Polymerized lenses that have not been swelled or otherwise equilibrated are considered cured lenses for purposes of this invention.

Methods of the invention may include additional solution treatment steps. For example, a step of rinsing the lenses of step (b) may be added. Further, a step of rinsing the lenses of step (b) followed by removing those lenses from the rinsing solution and placing those lenses in the solution of step (c)

Further the invention includes an antimicrobial ionic lens comprising a metal salt prepared by a method comprising the steps of
(a) treating an cured ionic lens for a sufficient period of time, with a first solution wherein the pH of said first solution is equal to the pKa of the ionic monomers, that were cured to form said ionic lens;
(b) adding a metal agent to said first solution and said cured lens after step (a)
(c) treating the lens of step (b) with a second solution comprising a salt precursor.

In order to illustrate the invention the following examples are included. These examples do not limit the invention. They are meant only to suggest a method of practicing the invention. Those knowledgeable in contact lenses as well as other specialties may find other methods of practicing the invention. However, those methods are deemed to be within the scope of this invention.

### EXAMPLES

The following abbreviations were used in the examples

Blue HEMA=the reaction product of reactive blue number 4 and HEMA, as described in Example 4 or U.S. Pat. No. 5,944,853
CGI 819=bis(2,4,6-trimethylbenzoyl)phenylphosphineoxide
DI water=deionized water

### DMA=N,N-dimethylacrylamide

HEMA=hydroxyethyl methacrylate
MAA=methacrylic acid;
mPDMS=mono-methacryloxypropyl terminated polydimethylsiloxane (MW 800-1000)
acPDMS=bis-3-acryloxy-2-hydroxypropyloxypropyl polydimethylsiloxane
Norbloc=2-(2′-hydroxy-5-methacrylyloxyethylphenyl)-2H-benzotriazole
ppm=parts per million micrograms of sample per gram of dry lens
PVP=polyvinylpyrrolidinone (360,000 or 2,500)
Simma 2=3-methacryloxy-2-hydroxypropyloxy)propylbis (trimethylsiloxy)methylsilane
TM=t-amyl alcohol

### Sodium Sulfate Packing Solution

deionized H₂O:
1.40 weight % sodium sulfate
0.185 weight % sodium borate [1330-43-4], Mallinckrodt
0.926 weight % boric acid [10043-35-3], Mallinckrodt
0.005 weight % methylcellulose

### Preparation Lens Type A

A hydrogel blend was made from the following monomer mix (all amounts were calculated as weight percent: 30.00% SIMM 2, 28.0% mPDMS, 5.0% acPDMS, 19.0% DMA, 7.15% HEMA, 1.60% MM, 7.00% PVP 360,000, 2.0% Norbloc, 1.0% CGI 819 and 0.02% Blue HEMA, 60 weight percent of the preceding component mixture was further diluted with diluent, 40 weight percent of 72.5:27.5 TAA:PVP 2,500, to form the final monomer mix. The blend placed in a two part contact lens mold and was cured using the following sequential conditions a) room temperature for 30 seconds using a visible light that emits 1 mW/sq cm, b) 75° C. 120 seconds, c) 75° C. 120 seconds 1.8 mW/sq/cm, and d) 75° C. 240 seconds 6.0 mW/sq cm. The cured lenses are removed from the molds and hydrated with IPA/DI water mixtures.

### Example 1

### Preparation of Antimicrobial Lenses from Cured Lenses without Buffer Treatment Step

Cured and hydrated lenses of Type A are placed in a jar with sodium iodide solution in deionized water (0.8 mL/lens) containing approximately methyl cellulose 100 ppm and rolled on a jar roller overnight (i.e. >8 hours). The lenses were transferred from the jar to a blister pack where the excess sodium iodide solution was removed. A solution (0.8 mL/lens) of silver nitrate in deionized water (concentration as per Table 1) was added to the blister for the time indicated in Table 1. The silver nitrate solution was removed, and the lenses were rinsed with deionized water and placed in sodium sulfate packaging solution. The blisters were sealed and autoclaved at 124° C. for 18 minutes and analyzed for silver content and haze using the method described below. The results are presented in Table 1.

Silver content of the lenses after lens autoclaving was determined by Instrumental Neutron Activation Analysis “INAA”. INAA is a qualitative and quantitative elemental analysis method based on the artificial induction of specific radionuclides by irradiation with neutrons in a nuclear reactor. Irradiation of the sample is followed by the quantitative measurement of the characteristic gamma rays emitted by the decaying radionuclides. The gamma rays detected at a particular energy are indicative of a particular radionuclide's presence, allowing for a high degree of specificity. Becker, D. A.; Greenberg, R. R.; Stone, S. F. J. Radioanal. Nucl. Chem. 1992, 160(1), 41-53; Becker, D. A.; Anderson, D. L.; Lindstrom, R. M.; Greenberg, R. R.; Garrity, K. M.; Mackey, E. A. J. Radioanal. Nucl. Chem. 1994, 179(1), 149-54. The INAA procedure used to quantify silver content in contact lens material uses the following two nuclear reactions:
1. In the activation reaction, ¹¹⁰Ag is produced from stable ¹⁰⁹Ag (isotopic abundance=48.16%) after capture of a radioactive neutron produced in a nuclear reactor.
2. In the decay reaction, ¹¹⁰Ag (τ^{1/2}=24.6 seconds) decays primarily by negatron emission proportional to initial concentration with an energy characteristic to this radio-nuclide (657.8 keV).
   The gamma-ray emission specific to the decay of ¹¹⁰Ag from irradiated. standards and samples are measured by gamma-ray spectroscopy, a well-established pulse-height analysis technique, yielding a measure of the concentration of the analyte.

The percentage of haze is measured using the following method. A hydrated test lens in borate buffered saline (SSPS) is placed in a clear 20×40×10 mm glass cell at ambient temperature above a flat black background, illuminating from below with a fiber optic lamp (Titan Tool Supply Co. fiber optic light with 0.5″ diameter light guide set at a power setting of 4-5.4) at an angle 66° normal to the lens cell, and capturing an image of the lens from above, normal to the lens cell with a video camera (DVC 1300C:19130 RGB camera with Navitar TV Zoom 7000 zoom lens) placed 14 mm above the lens platform. The background scatter is subtracted from the scatter of the lens by subtracting an image of a blank cell using EPIX XCAP V 1.0 software. The subtracted scattered light image is quantitatively analyzed, by integrating over the central 10 mm of the lens, and then comparing to a −1.00 diopter CSI Thin Lens®, which is arbitrarily set at a haze value of 100, with no lens set as a haze value of 0. Five lenses are analyzed and the results are averaged to generate a haze value as a percentage of the standard CSI lens.

TABLE 1

| Nal | | AgNO3 | | | |
|---|---|---|---|---|---|
| Concentration | | Concentration | AgNO3 Time | Silver Dose | Haze (% |
| (ppm) | Nal Time | (ppm) | (minutes) | (mcg) | vs. CSI) |
| 5000 | Overnight | 1000 | 2 | 32.0 | 108 |
| 7500 | Overnight | 1000 | 2 | 38.1 | 117 |
| 10000 | Overnight | 1000 | 2 | 42.6 | 161 |
| 5000 | Overnight | 1000 | 2 | 30.3 | 84 |
| 7500 | Overnight | 1000 | 2 | 36.0 | 105 |
| 10000 | Overnight | 1000 | 2 | 40.8 | 139 |
| 5000 | Overnight | 1000 | 2 | 31.7 | 86 |
| 7500 | Overnight | 1000 | 2 | 38.4 | 119 |
| 10000 | Overnight | 1000 | 2 | 39.9 | 146 |
| 2800 | Overnight | 300 | 5 | 15.8 | 58 |
| 2800 | Overnight | 300 | 5 | 15.0 | 67 |
| 2800 | Overnight | 300 | 5 | 16.0 | 65 |
| 2800 | Overnight | 300 | 5 | 16.0 | 76 |
| 2800 | Overnight | 300 | 5 | 15.9 | 74 |
| 2800 | Overnight | 300 | 5 | 15.3 | 64 |
| 2800 | Overnight | 300 | 5 | 14.5 | 56 |

### Example 2

### Preparation of Antimicrobial Lenses from Cured Lenses with Buffer Treatment

Cured and hydrated lenses of Type A were placed in a halide free borate buffered deionized water with a pH of 7.4 (1.6 mL/lens). The lenses were maintained in this solution overnight. The lenses were removed from this solution and placed into a container with approximately 0.800 mL of silver nitrate solution in a concentration as stated in Table 2. The lenses remained in that solution for the time indicated and rinsed with about 100 μL of deionized water. The lenses were placed in approximately 0.800 mL of sodium iodide solution/deionized water in a concentration and for a time as per Table 2. The treated lenses were transferred to sodium sulfate packaging solution (0.800 mL) sealed and heated to approximately 124° C. for about 18 minutes to sterilize the lenses. The haze level and the silver content of the lenses were determined by the methods described in Example I.

TABLE 2

| | AgNO3 | AgNO3 | | Nal | | | |
|---|---|---|---|---|---|---|---|
| Staged in | Concentration | Time | AgNO3 | Concentration | Nal Time | Silver Dose | Haze (% |
| Buffer? | (ppm) | minutes | Rinse Step? | (ppm) | minutes | (mcg) | vs. CSI) |
| No | 500 | 15 | No | 1000 | 5 | 4.8 | 20.8 |
| No | 150 | 90 | No | 5000 | 2 | 2.9 | 24.0 |
| No | 150 | 15 | Yes | 5000 | 5 | 2.1 | 16.8 |
| No | 500 | 90 | Yes | 1000 | 2 | 2.4 | 13.0 |
| Overnight | 150 | 90 | No | 1000 | 5 | 41.4 | 44.8 |
| Overnight | 500 | 90 | Yes | 5000 | 5 | 91.1 | 147.2 |
| Overnight | 150 | 15 | Yes | 1000 | 2 | 28.0 | 34.0 |
| Overnight | 500 | 15 | No | 5000 | 2 | 75.4 | 56.8 |
| Overnight | 150 | 65 | No | 1100 | 1 | 39.3 | 28.0 |
| Overnight | 100 | 15 | No | 1100 | 1 | 20.9 | 23.2 |
| Overnight | 125 | 40 | No | 1000 | 2 | 35.8 | 28.0 |
| Overnight | 100 | 15 | No | 900 | 3 | 25.4 | 22.1 |
| Overnight | 150 | 65 | No | 900 | 3 | 45.5 | 35.2 |
| Overnight | 150 | 65 | Yes | 1100 | 3 | 46.4 | 29.2 |
| Overnight | 150 | 65 | Yes | 900 | 1 | 34.9 | 28.2 |
| Overnight | 100 | 15 | Yes | 1100 | 3 | 25.3 | 20.7 |
| Overnight | 125 | 40 | Yes | 1000 | 2 | 33.6 | 23.4 |
| Overnight | 100 | 15 | Yes | 900 | 1 | 19.0 | 20.1 |
| Overnight | 100 | 65 | No | 1100 | 3 | 33.7 | 25.0 |
| Overnight | 100 | 65 | No | 900 | 1 | 24.7 | 21.6 |
| Overnight | 150 | 15 | No | 1100 | 3 | 31.2 | 20.8 |
| Overnight | 125 | 40 | No | 1000 | 2 | 33.5 | 30.8 |
| Overnight | 150 | 15 | No | 900 | 1 | 25.6 | 23.8 |
| Overnight | 125 | 40 | Yes | 1000 | 2 | 32.4 | 34.4 |
| Overnight | 100 | 65 | Yes | 1100 | 1 | 24.8 | 15.7 |
| Overnight | 150 | 15 | Yes | 1100 | 1 | 25.3 | 18.2 |
| Overnight | 100 | 65 | Yes | 900 | 3 | 31.4 | 21.5 |
| Overnight | 150 | 15 | Yes | 900 | 3 | 34.3 | 22.7 |

The data in Tables 1 and 2 illustrates that lenses produced by the methods of the invention contain more silver with lower haze numbers.

What is claimed is:

1. A method of preparing an ionic antimicrobial lens comprising a metal salt, wherein said method comprising the steps of
(a) treating an cured ionic lens for a sufficient period of time, with a first solution wherein the pH of said first solution is equal to the pKa of the ionic monomers, that were cured to form said ionic lens;
(b) adding a metal agent to said first solution and said cured lens after step (a)
(c) treating the lens of step (b) with a second solution comprising a salt precursor.

2. The method of claim 1 wherein the pH of the first solution is at least about one unit greater than the pKa.

3. The method of claim 1 wherein the pH of the first solution is least about two units greater than the pKa.

4. The method of claim 1 wherein the pH of the first solution is about pH 6 to about pH 8.

5. The method of claim 1 wherein the metal agent is selected from the group consisting of silver nitrate, silver triflate, or silver acetate, silver tetrafluoroborate, silver sulfate, zinc acetate, zinc sulfate, copper acetate, and copper sulfate.

6. The method of claim 1 wherein the metal agent is silver nitrate.

7. The method of claim 1 wherein the salt precursor is selected from the group consisting of sodium chloride, sodium iodide, sodium bromide, lithium chloride, lithium sulfide, sodium sulfide, potassium sulfide, potassium iodide, and sodium tetrachloro argentite.

8. The method of claim 1 wherein the salt precursor is sodium iodide.

9. The method of claim 1 wherein the ionic lens is selected from the group consisting of etafilcon A, balafilcon A, bufilcon A, deltafilcon A, droxifilcon A, phemfilcon A, ocufilicon A, perfilcon A, ocufilcon B, ocufilcon C, ocufilcon D, ocufilcon E, metafilcon A, B, vifilcon A focofilcon A, and tetrafilcon B.

10. An antimicrobial ionic lens comprising a metal salt prepared by a method comprising the steps of
(a) treating an cured ionic lens for a sufficient period of time, with a first solution wherein the pH of said first solution is equal to the pKa of the ionic monomers, that were cured to form said ionic lens;
(b) adding a metal agent to said first solution and said cured lens after step (a)
(c) treating the lens of step (b) with a second solution comprising a salt precursor.

11. The antimicrobial lens of claim 10 wherein the pH of the first solution is at least about one unit greater than the pKa.

12. The antimicrobial lens of claim 10 wherein the pH of the first solution is least about two units greater than the pKa.

13. The antimicrobial ionic lens of claim 10 wherein the pH of the first solution is about pH 8.

14. The antimicrobial ionic lens of claim 10 wherein the metal agent is selected from the group consisting of silver nitrate, silver triflate, or silver acetate, silver tetrafluoroborate, silver sulfate, zinc acetate, zinc sulfate, copper acetate, and copper sulfate.

15. The antimicrobial ionic lens of claim 10 wherein the metal agent is silver nitrate.

16. The antimicrobial ionic lens of claim 10 wherein the salt precursor is selected from the group consisting of sodium chloride, sodium iodide, sodium bromide, lithium chloride, lithium sulfide, sodium sulfide, potassium sulfide, potassium iodide, and sodium tetrachloro argentite.

17. The antimicrobial ionic lens of claim 10 wherein the salt precursor is sodium iodide.

18. The antimicrobial ionic lens of claim 10 wherein the ionic lens is selected from the group consisting of etafilcon A, balafilcon A, bufilcon A, deltafilcon A, droxifilcon A, phemfilcon A, ocufilicon A, perfilcon A, ocufilcon B, ocufilcon C, ocufilcon D, ocufilcon E, metafilcon A, B, vifilcon A focofilcon A, and tetrafilcon B.

19. The antimicrobial ionic lens of claim 10 wherein the metal salt is silver iodide.
